(19) United States
(12) Patent Application Publication
(10) Pub. No.: US 2025/0082598 A1
(43) Pub. Date: Mar. 13, 2025

(54) Melafenoxate, 2-(1-adamantylamino)ethyl 2-(4-chlorophenoxy)acetate and derivatives thereof for treating circadian rhythm sleep disorders with neurodegenerative diseases such as Alzheimer's disease

(71) Applicant: NLS Pharmaceutics AG, Zurich (CH)

(72) Inventor: Eric KONOFAL, Senlis (FR)

(73) Assignee: NLS Pharmaceutics AG, Zurich (CH)

(21) Appl. No.: 18/576,990

(22) PCT Filed: Jul. 8, 2022
(86) PCT No.: PCT/EP2022/069204

§ 371 Date: Jan. 5, 2024

(30) Foreign Application Priority Data
Jul. 8, 2021 (EP) 21305942.1

(51) Int. Cl. A61K 31/221; A61P 25/28

(52) U.S. Cl. CPC A61K 31/221; A61P 25/28

(57) ABSTRACT

The invention concerns a compound of formula (I) R₁=H or acyl group, preferably R₁=H; R₂=halogen atom selected in the group consisting of: F, Cl, Br, I, preferably R₂=Cl or a pharmaceutically acceptable isomer, salt and/or solvate thereof, for use in preventing and/or treating circadian rhythm sleep disorders and/or circadian rhythm sleep neurological diseases and/or any medical condition associated due to sleep deprivation.

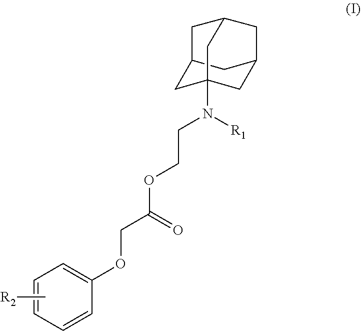

### FIELD OF THE PRESENT INVENTION

The present invention relates to the use of Melafenoxate, 2-(1-adamantylamino)ethyl 2-(4-chlorophenoxy) acetate and derivatives thereof in the prevention and/or treatment of circadian rhythm sleep disorders and/or circadian rhythm sleep neurological diseases and/or any medical condition associated due to sleep deprivation and/or circadian rhythm disruption, preferably with Alzheimer's disease.

### BACKGROUND OF THE PRESENT INVENTION

Circadian rhythms are ubiquitous in all living organisms and nearly all physiological functions, most notably sleep and wake cycles, exhibit circadian rhythmicity. Circadian rhythms are endogenous and persist in the absence of environmental time cues¹.

The sleep-wake cycle is regulated by a complex interaction between the homeostatic process (a drive for sleep which builds up during wakefulness and declines during sleep) and circadian process (a sleep-wake independent 24-hour oscillatory rhythm that modulates sleep propensity). The circadian drive for sleep is the highest at the end of biological night and lowest at the end of biological day. In the entrained situation, when homeostatic drive for sleep dissipates with sleep, the circadian drive for sleep increases in a compensatory manner to facilitate the consolidation of sleep. Conversely, when homeostatic drive for sleep increases with wakefulness during the biological day, the circadian drive for sleep decreases and helps the consolidation of wakefulness².

Circadian rhythm sleep disorders (CRSD) arise from a chronic or recurrent pattern of sleep and wake disturbance that is due to dysfunction of the circadian clock system, or misalignment between the timing of the endogenous circadian rhythm and externally imposed social and work cycles, that result in clinically significant functional impairments.

Alzheimer's disease (AD) is characterized by progressive loss of memory and other cognitive functions that severely impact the patients' social skills and ability to perform a routine task. Presence of amyloid plaques, hyperphosphorylated tau protein in the patients' brains are the hallmarks of AD.

More than 50 million people globally are living with dementia, and AD accounts for 70% of the cases. This figure is predicted to double every twenty years³. The rising numbers stem from a dire lack of effective treatment. Therefore, it is crucial to identify and understand contributing factors to the AD pathology, which may be modified to manage and slow its progression at the early stages.

Circadian rhythm sleep disorders are commonly observed in Alzheimer's patients from the early stages of the disease³⁻⁵. Moreover, postmortem analysis of the brains confirms morphological changes in the core machinery of the central circadian clock⁶. The clinical evidence of circadian rhythm disruption and AD association is overwhelming. However, whether circadian rhythm disruption is a cause or the consequence of AD or which role melatonin plays in the pathophysiology of AD is not fully elucidated or understood⁷.

Melatonin (N-acetyl-5-methoxytryptamine) is a multifunctional neurohormone of the pineal gland that is excreted into the blood and cerebrospinal fluid (CSF) and plays an important role in the regulation of seasonal and circadian rhythms, a key of most treatment used to treat CRSD⁸.

Melatonin production is decreased with aging and in certain diseases, including neurodegenerative diseases, indicating that the deregulation of melatonin may cause the development or progress of human diseases⁹⁻¹¹. CSF melatonin levels are significantly reduced to levels one-half the levels of young individuals in aged populations with early Alzheimer's disease-like neuropathological changes in the brain^{11,12}. In fact, it has been confirmed that removing the pineal gland results in hippocampal deformities that are reversed by oral administration of melatonin¹³. Common indications of melatonin reduction in Alzheimer's disease (AD) patients are sleep disruptions, nightly restlessness, and sundowning, all of which are more frequently observed in the elderly population than in other populations and particularly in patients with AD¹⁴.

In addition to its role in sleep and circadian rhythms, melatonin has been shown to exert neuroprotective effects, antioxidant defense, anti-inflammatory effects, decreases cholinesterase activity and prevents mitochondrial damage and apoptosis and anti-apoptotic activity in both cellular and animal models of AD¹⁵⁻¹⁶.

Increased expression of anti-oxidative enzymes and proteins are the key markers of oxidative stress. The observation shows that such proteins are present in the neuropathological lesions of AD patients that supports the possible involvement of oxidative stress in pathogenic mechanisms in this disorder¹⁰.

CRSD are increasingly reported from the early stages of AD: sleep deprivation and insomnia are associated with the pathogenesis of AD and may have an impact on the symptoms and development; sleep deprivation impairs glymphatic bulk flow through brain parenchyma, impairing clearance of β-amyloid and tau; circadian rhythm disruption leads to increased oxidative stress in neurons; sleep deprivation and circadian rhythm disruption abolishes neuroprotective effect of melatonin; sleep deprivation and circadian rhythm disruption induce pathological stress granule to aggravate tau aggregation.

According to the international classification system, CRSD include Circadian Rhythm Sleep Disorder, Irregular Sleep-Wake Type G47.23 (ICD-10); Circadian Rhythm Sleep Disorder, Irregular Sleep-Wake Type 307.45-3 (DSM-5); and Circadian Rhythm Sleep-Wake Disorder, Irregular Sleep-Wake Rhythm Disorder 307.45-3 (ICSD-3)¹⁷⁻¹⁹.

The central disorders of hypersomnolence (CDH) are marked by pathologic daytime sleepiness and/or unappropriated arousal status. The International Classification of Sleep Disorders, Third Edition (ICSD-3), classifies eight different Central Disorders of Hypersomnolence: Narcolepsy Type 1, Narcolepsy Type 2, Idiopathic Hypersomnia, Kleine-Levin syndrome, hypersomnia associated with a psychiatric disorder, hypersomnia due to a medical disorder, hypersomnia due to a medication or substance, and insufficient sleep syndrome¹⁷.

The underlying pathophysiology of these disorders is unclear.

Kleine-Levin Syndrome (KLS) is an orphan disease characterized by recurrent, relapsing-remitting episodes of severe hypersomnia, a need for excessive amounts of sleep (hypersomnolence), (i.e. for 18 to 20 hours per day); excessive food intake (compulsive hyperphagia, binge-eating); and cognitive impairment, apathy, derealization, altered mood and behavioral changes such as an abnormally uninhibited sexual drive.

The disorder primarily affects adolescent males who appear to be affected three times as often as females and usually around the age of 16 years. When awake, affected individuals may exhibit irritability, lack of energy (lethargy), and/or lack of emotions (apathy). They may also appear confused (disoriented) and experience hallucinations with psychosis state.

Symptoms of KLS are cyclical. An affected individual may go for weeks or months without experiencing symptoms. When present, symptoms may persist for days to weeks.

The exact cause of KLS is not known. However, researchers believe that in some cases, hereditary factors may cause some individuals to have a genetic predisposition of developing this disorder. It is thought that symptoms of KLS may be related to malfunction of the portion of the brain that helps to regulate functions such as sleep, appetite, and body temperature (hypothalamus).

Recent findings reinforce the idea there is hypometabolism in the mesotemporal cortex, including the hippocampus, in almost half of patients with KLS, and that the severity of these hypometabolisms are markers of a more recent disorder.

Previously, some researchers speculated that KLS may be an autoimmune disorder as NT1. Less than 500 cases have been reported in the medical literature. However, because cases of KLS often go unrecognized, the disorder is under-diagnosed, making it difficult to determine its true frequency in the general population. Many symptomatic and prophylactic treatments have been offered to patients, without controlled trials and only based on case reports. One of the various stimulants tried, Amantadine, a drug with dopamine reuptake inhibitory stimulant and antiviral properties, had probably the most significant response, found to be 41% compared to rest of stimulants as reported by Arnulf et al. in a systematic review comparing all proposed treatments of KLS.

Its mechanism of action is still unclear. Amantadine increases dopamine synthesis and release, blocks presynaptic dopamine reuptake, and act as an NMDA glutamatergic receptor antagonist. The effect was often lost in subsequent episodes²⁰. In our case the excellent response was maintained even in the second episode, but the lack of perspective is limiting to make early conclusions. Amantadine, an interesting choice of rapidly efficacious treatment that can be considered in particular episodes of KLS but the necessity of further studies is still needed and no definitive treatment for KLS during the episode as well as inter-episodic period is recommended. There is evidence of reduced dopaminergic tone during periods of hypersomnolence in KLS, and that the dopamine D4 receptor gene (DRD4) may be particularly implicated in binge-eating that might lead to obesity as recently published.

Irregular Sleep-Wake Rhythm Disorder (ISWRD) is an uncommon circadian rhythm sleep disorder. Subjects with ISWRD are sleeping intermittently and irregularly during the day in addition to not sleeping continuously at night. ISWRD disturbances are therefore sometimes referred to as fragmentation of the normal circadian, or 24-hour, diurnal pattern of sleep and wake²⁰.

ISWRD could be identified in early stages of AD. Subject with ISWRD also experience excessive daytime sleepiness and other cognitive impairments while they are awake. ISWRD is commonly seen in children with neurodevelopmental disabilities, including Angelman, Williams, and Smith-Magenis syndromes⁸. ISWRD is commonly seen in elderly individuals with neurodegenerative disorders, such as dementia and Alzheimer's disease (AD)²¹.

Melatonin and its receptors (MT₁ and MT₂, belonging as they do to G-protein coupled receptors, GPCR) have attracted attention. This is due to some reports suggesting a key role for this monoamine. Among these are those reporting that melatonin might be an interesting biomarker, showing an inverse correlation between the levels of melatonin in cerebrospinal fluid and the severity of the neuropathology²². However, there also are authors who report melatonin as a potential therapeutic agent because it can ameliorate the formation of Aβ-plaques and neurofibrillary tangles²³.

Moreover, melatonin administration improves the behavioral impairments related to AD in murine models, including disrupted circadian rhythm, cognition, learning, memory, motor function, mood, sleep, and stress response^{24,25}. Furthermore, melatonin induces beneficial effects on the cholinergic system by increasing acetylcholine release and inhibiting choline acetyltransferase²⁵. Furthermore, it also appears to exert benefits linked to the modulation of other monoamine systems, such as the serotonergic and dopaminergic systems by means of unestablished mechanisms^{23,25}.

Additionally, the chemical structure of melatonin is related to structures acting as antioxidants or regulators of inflammation. In fact, its administration limits the excessive influx of Ca²⁺ and the excessive efflux of Mg²⁺ related to some inflammatory and neurotoxicity mechanisms^{23,26}.

In Kleine-Levin Syndrome (KLS), during hypersomnia attack, the amplitude of the Circadian Rest-Active Rhythms drastically decreases and decreases inter-daily stability²⁷.

Critchley and Hoffman actually defined the syndrome in 1942 and gave it its name after the publication of the classic paper, “The Syndrome of Periodic Somnolence and Morbid Hunger (Kleine-Levin Syndrome).” It predominantly affects adolescent males with episodes usually lasting up to a few weeks and result in complete recovery. Those who are affected are asymptomatic between these episodes; however, multiple relapses may occur. Each episode can last from a week to 1 to 2 months. The duration of KLS can vary based on the clinical manifestation of the syndrome²⁸.

Recent findings suggest a relationship between KLS, circadian regulation, and Bipolar Disorder, and indicate that the TRANK1 gene polymorphisms in conjunction with reported birth difficulties may predispose to KLS²⁹. Also TRANK1 gene seems biologically related to dendritic spine, synaptic plasticity, axon guidance and circadian entrainment processes, and are also more likely to exhibit strong associations in psychiatric genomewide association studies (e.g., the CACNA1C gene which is affected in the regulation of sleep in early development)³⁰. If melatonin has several advantages of clinical value, extensive review of literature revealed that melatonin is not sufficiently effective in treating most CRSD. Some of the reasons for a limited efficacy of this natural hormone are related to its extremely short half-life in the circulation, and to the fact that sleep maintenance is also regulated by mechanisms downstream of primary melatoninergic actions. The development of melatonin receptor agonists with a longer half-life is an urgent need which could be suitable for a successful treatment of CRSD and related disorders³⁰.

Despite the cloning of the melatonin receptors in the mid-1990s, research into the pathophysiological role of melatonin 1 (MT₁) receptor in the brain has been hampered by the lack of selective ligands for this melatonin receptor subtype. This lack of MT₁ selective ligands can be attributed, in part, to the fact that human MT₁ and MT₂ receptors share about 60% of their amino-acid sequence within the transmembrane domains, and also due to the absence of their crystal structures³¹.

A decrease of MT₁ melatonin receptor levels in the individuals with the genetic risk variant increases the amyloidogenic processing of amyloid precursor protein-β (APPβ) in neurons and enhances the pathological process of Alzheimer Disease (AD).

Above all, it has been reported that amyloidogenic processing of APP was increased in cells where the level of MT₁ₐ melatonin receptor was decreased, as shown by the 40% increase in the level of soluble APPβ³².

A model of mechanistic explanations for the association of rs12506228 linked to decreased MTNR1A (gene of MT₁ receptor expression in brain) both with AD and intolerance to shift work has been hypothesized³².

Previously, it has been shown that melatonin reduces neuronal cell death along with the preservation and activation of MT₁ in an HTT (Huntingtin) mutant cell model. Furthermore, the study showed that MT₁ level was lower in Huntington's disease (HD) mice than in wild-type mice³³. In addition, a reduction in the MT₁ level in the amygdala and substantia nigra of the brain could lead to Parkinson's Disease (PD)³⁴. However, a selective melatonin receptor MT₁ agonist able to alleviate PD or HD symptomatology and prevent their neuronal apoptosis has never been studied.

Also, it has been revealed that melatonin decreases the levels of IL-17 (interleukin involved in Multiple Sclerosis) secreted from TH17 cells via an MT₁-dependent pathway³⁵.

Moreover, it has also been shown that the levels of melatonin and MT₁ but not MT₂ are much lower in the spinal cord of Amyotrophic Lateral Sclerosis (ALS) mice than those in wild-type mice³⁶. Thus, the antiapoptotic effect of melatonin in ALS may be dependent on the MT₁ pathway.

ALS is characterized by the progressive and selective degeneration of motor neurons (MNs) in the brain stem, hypoglossal motor neurons (HMNs), facial motor neurons (FMNs) and the spinal cord, resulting in progressive paralysis and eventual death. It has been reported that melatonin not only effectively delays the progression and mortality of the disease but also significantly inhibits motor neuron death by inactivating the receptor interacting protein-2 (Rip2)/caspase-1 pathway and caspase-3 and blocking the release of mitochondrial cytochrome c in a mutant superoxide dismutase 1 (SOD1) (G93A) transgenic mouse model of ALS. The protective effect of melatonin on apoptosis in ALS was shown to be related to the inhibition of the caspase-1/cytochrome c/caspase-3 pathway³³.

On circadian rhythm and arousal, the relaxin-3/RXFP3 signaling promote a range of consummatory behaviors is in line with its likely primary role in driving arousal and motivated behavior more broadly³⁷⁻³⁹.

Relaxin family peptide receptor 3 (relaxin-3/RXFP3), a G protein-coupled receptor (GPCR) implicated in stress responses, feeding and metabolism, motivation, reward, and sexual behavior has been proposed to modulate emotional-behavioral functions such as arousal and behavioral activation, appetite regulation, stress responses, anxiety, memory, sleep and circadian rhythm³⁹.

RXFP3 as a potential therapeutic target for treatment of neuroendocrine disorders and related behavioral dysfunction⁴⁰, but none evidence based on relaxin-3/RXFP3 system and KLS has been reported.

Melafenoxate is the first selective melatonin 1 (MT₁) receptor agonist found. Considering its unique binding profile, the inventors postulate its potential benefit on circadian rhythm sleep disorders and/or circadian rhythm sleep neurological diseases and/or any medical condition associated due to sleep deprivation and/or circadian rhythm disruption and/or central disorders of hypersomnolence.

Since previous studies have identified that melatonin treatment ameliorated the scopolamine-induced overactivation of astrocytes and microglial cells and reduced the overexpression of TNF-α and IL-1β, preventing neuroinflammation and memory impairment in the scopolamine-administered adult mice¹⁰, animal models of scopolamine-induced cognitive impairment are widely used to study underlying mechanisms and treatment of cognitive impairment in neurodegenerative diseases such as Alzheimer's disease (AD)^{41,42}.

Scopolamine, a nonselective antimuscarinic agent, leads to progressive impairment of learning and memory principally by blocking central cholinergic signaling⁴³⁻⁴⁵. It is a well-known phenomenon that scopolamine generates reactive oxygen species and results in oxidative stress, leading to impairments in memory and cognitive function, as seen in patients with AD^{46,47}.

Some inhibitors of acetylcholine esterase (AChE) such as lactucopicrin⁴⁷ or well-known donepezil⁴⁸ or pharmacological agent acting acethylcholinergic neurons have been found to abate the scopolamine-induced brain injury, but none pharmacological agent targeting selectively on melatoninergic type 1 receptor alone, has been found efficient on this model.

### SUMMARY OF THE PRESENT INVENTION

An object of the invention is a compound of formula (I)

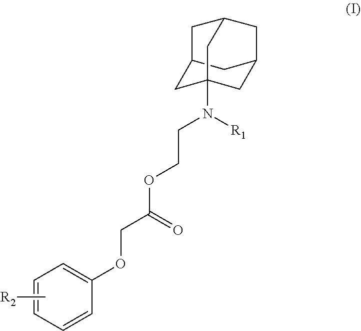

R₁=H or acyl group
R₂=halogen atom selected in the group consisting of: F, Cl, Br, I,
or a pharmaceutically acceptable isomer, salt and/or solvate thereof, for use in preventing and/or treating circadian rhythm sleep disorders and/or circadian rhythm sleep neurological diseases and/or any medical condition associated due to sleep deprivation and/or circadian rhythm disruption, preferably with Alzheimer's disease.

Another object of the invention is a pharmaceutical composition comprising a compound of formula (I) or a pharmaceutically acceptable isomer, salt and/or solvate thereof as defined before and a pharmaceutically acceptable excipient for use preventing and/or treating circadian rhythm sleep disorders and/or neurological diseases and/or any medical condition associated due to sleep deprivation and/or circadian rhythm disruption, preferably with Alzheimer's disease.

### FIGURES

FIG. 1 Effects of NLS-8 on locomotor activity in open-field. Differences vs. Control group: no significant (ns) in all cases.

FIG. 2 Effects of scopolamine and of scopolamine+NLS-8 on the discrimination index. Difference: vs. 0: #p≤0.05, ###p≤ 0.001; differences vs. Control group: *p≤0.05; differences NLS-8 groups vs Scopo group: not significant (ns) in all cases.

FIG. 3 Effects of scopolamine and of scopolamine+NLS-8 on the exploration time of the two objects during the choice trial. Differences vs. Control group: *p≤ 0.05; differences vs. Scopo group: § p≤0.05.

### DETAILED DESCRIPTION OF THE PRESENT INVENTION

The first subject-matter of the invention relates to a compound of formula (I)

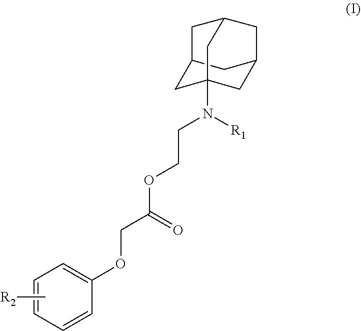

R₁=Hor or acyl group, preferably R₁=H
R₂=halogen atom selected in the group consisting of: F, Cl, Br, I, preferably R₂=Cl
or a pharmaceutically acceptable isomer, salt and/or solvate thereof, for use in preventing and/or treating circadian rhythm sleep disorders and/or circadian rhythm sleep neurological diseases and/or any other medical conditions associated due to sleep deprivation and/or circadian rhythm disruption, preferably with Alzheimer's disease.
R₂ is in ortho, meta or para position, preferably in para position.
Formula (I) has a chiral center.
Thus, “isomer” means “enantiomer”.

According to the present invention, and when not specified otherwise, the term “compound of formula (I)” refers to compound of formula (I) in its racemic form or in its enantiomeric forms.

An “optically pure compound of formula (I)” means an enantiomer in an enantiomeric excess of more than 95%, preferably of more than 96%, more preferably of more than 97%, even more preferably of more than 98%, particularly preferably of more than 99%.

When R1=H and R2=Cl in para position, compound of formula (I) is Melafenoxate, 2-(1-adamantylamino)ethyl 2-(4-chlorophenoxy)acetate, a 1:1 racemic mixture and its R- and S-enantiomers, their salts, in particular their hydrochloride salt.

Compound of formula (I) is preferably used at a therapeutic dose comprised between 0.1 mg/kg/day and 100 mg/kg/day is administrated to a patient in need thereof, more preferably between 0.5 and 50 mg/kg/day.

The second subject-matter of the invention relates to a method of prevention and/or treatment of circadian rhythm sleep disorders and/or circadian rhythm sleep neurological diseases and/or any medical condition associated due to sleep deprivation and/or circadian rhythm disruption, preferably with Alzheimer's disease comprising the administration of a compound of formula (I) as defined above or a pharmaceutically acceptable isomer, salt and/or solvate thereof, to a patient in need thereof.

The third subject-matter of the invention relates to a pharmaceutical composition comprising a compound of formula (I) or a pharmaceutically acceptable isomer, salt and/or solvate thereof as defined above and a pharmaceutically acceptable excipient for use in preventing and/or treating circadian rhythm sleep disorders and/or circadian rhythm sleep neurological diseases and/or any medical condition associated due to sleep deprivation and/or circadian rhythm disruption, preferably with Alzheimer's disease.

Preferably, the pharmaceutical composition for use according to the invention comprises between 1 mg to 800 mg, preferably 20 mg to 400 mg of compound of formula (I).

Preferably, the pharmaceutical composition for use according to the invention is suitable for oral administration, for example in the form of a tablet, a capsule, a syrup, a solution, a powder or parenteral administration, for example in the form of a solution, such as an injectable solution and for TDS (transdermal delivery systems).

The fourth subject-matter of the invention relates to a method of prevention and/or treatment of circadian rhythm sleep disorders and/or circadian rhythm sleep neurological diseases and/or any other medical conditions associated due to sleep deprivation and/or circadian rhythm disruption, preferably Alzheimer's disease comprising the administration of a pharmaceutical composition as defined above to a patient in need thereof.

Preferably, circadian rhythm sleep disorders are selected in the group consisting of Circadian Rhythm Sleep Disorder, Irregular Sleep-Wake Type G47.23 (ICD-10); Circadian Rhythm Sleep Disorder, Irregular Sleep-Wake Type 307.45-3 (DSM-5); and Circadian Rhythm Sleep-Wake Disorder and Irregular Sleep-Wake Rhythm Disorder 307.45-3 (ICSD-3).

Preferably, said circadian rhythm sleep neurological diseases are selected in the group of:
1) neurodegenerative disorders including dementia, Alzheimer's disease (AD), Huntington's disease (HD), Parkinson's Disease (PD) and Amyotrophic Lateral Sclerosis (ALS).
2) neurodevelopmental disabilities including Angelman, Williams, and Smith-Magenis syndromes.

### Example

Melafenoxate was prepared as shown below, in 4 steps.

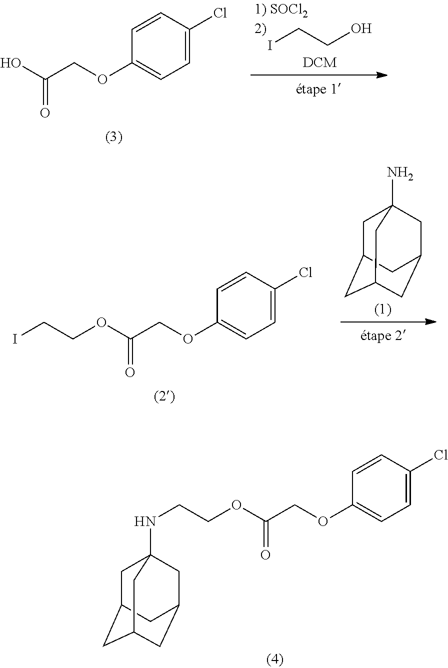

Melafenoxate was tested at 10⁻⁵ M, calculated as a % inhibition of control specific binding of a radioactively labeled ligand specific for each target.

This binding profile panel was broadly defined with roughly an equal number of selective, central and peripheral therapeutically relevant targets, including native animal tissues, radioligands and specific enzymes involved in cell cycle regulation in accordance with Eurofins Standard Operating Procedure.

For radioligand binding experiments, the half maximal inhibitory concentration (IC₅₀) and the half maximal effective concentration (EC₅₀) values were determined (via computer software) by nonlinear regression analysis of the competition curves using Hill equation curve fitting. The inhibition constants (Kᵢ) were calculated using the Cheng-Prusoff equation (Kᵢ=IC₅₀/(1+(L/K_{D})), where L is the concentration of radioligand in the assay, and K_{D} is the affinity of the radioligand for the receptor.⁴⁹

The results are expressed as a % control specific binding ([measured specific binding/control specific binding]×100) and as a % inhibition of control specific binding (100−[(measured specific binding/control specific binding)×100]) obtained in the presence of the test compounds.

Melafenoxate was tested in a battery of additional assays for inhibition of radioligand binding by CEREP (Eurofins, France) that included human A1, A2A, and A3 adenosine receptors, α1- and α2-adrenergic receptors, human β1-adrenergic receptor, human AT1 angiotensin receptor, benzodiazepine receptor, human bradykinin receptor, human CCK1 cholecystokinin receptor, human D1 and D2 dopamine receptors, human endothelin receptor type A, GABAA receptor, human galactose transporter, human CXC chemokine receptors, human C-C chemokine receptor type 1, H1 and H2 histamine receptors, human MC4 melanocortin receptor, MT₁, MT₂ melatonin receptors, human M1, M2, and M3 muscarinic acetylcholine receptors, human NK1, NK2 and NK3 neurokinin receptors, human Y1 and Y2 neuropeptide receptors, human NTS1 neurotensin receptor, human μ-, δ-, and κ-opioid receptors and opioid-like receptor, human 5-HT1A, 5-HT1B, 5-HT2A, 5-HT3, 5-HT5A, 5-HT6, and 5-HT7 serotonin receptors, somatostatin receptor, human vasoactive intestinal peptide receptor, human vasopressin receptor, Ca²⁺ channel, K+v channel, SK+Ca channel, Na⁺ channel, and Cl⁻ channel.

Results showing an inhibition (or stimulation) lower than 25% are not considered significant and mostly attributable to variability of the signal around the control level. Low to moderate negative values have no real meaning and are attributable to variability of the signal around the control level.

An inhibition or stimulation of more than 50% is considered a significant effect of the test compounds and between 25% and 50% indicated of weak to moderate effects that should be confirmed by further testing as they are within a range where more inter-experimental variability can occur.

Fifty percent is a common cut-off for further investigation (i.e. determination of IC₅₀ or EC₅₀ values from concentration-response curves).

Significant or pertinent findings of these binding assays are respectively presented for Melafenoxate in Table 1.

TABLE 1
Binding activity sites for Melafenoxate

| | % Inhibition at |
|---|---|
| | 10⁻⁵ M |
| Assay | Melafenoxate |
| MT₁ (MLT₁ₐ) (h) (agonist radioligand) | 75.2 |
| NK2 (h) (agonist radioligand) | 33.5 |
| DAT (h) (antagonist radioligand) | 20.9 |
| NET (h) (antagonist radioligand) | 16.5 |
| Cl— channel (GABA-gated) (antagonist radioligand) | 13.5 |
| 5-HT7 (h) (agonist radioligand) | 12.8 |
| 5-HT1A (h) (agonist radioligand) | 10.4 |

Melafenoxate is the first melatonin type 1 (MT₁) receptor agonist found. Its unique binding profile (Table 1) suggests its potential benefit on circadian rhythm sleep disorders and/or neurological diseases and/or any other medical condition associated due to sleep deprivation and/or circadian rhythm disruption.

Melafenoxate as melatonin 1 (MT₁) receptor agonist identified can be used to prevent and/or to treat circadian rhythm sleep disorders (CRSD) and/or neurological diseases as neurodegenerative disorders including dementia, Alzheimer's Disease (AD), Huntington's disease (HD), Parkinson's Disease (PD), Amyotrophic Lateral Sclerosis (ALS), neurodevelopmental disabilities as Angelman, Williams and Smith-Magenis syndromes, and/or any other medical conditions associated due to sleep deprivation and/or circadian rhythm disruption.

Another pertinent finding for the mechanism of action of Melafenoxate involved relaxin family receptors (RXFP3 and RXFP4).

Relaxin-3/RXFP3 and relaxin-4/RXFP4 receptors are involved in neuromodulatory system and the potential therapeutic targets for neuropsychiatric disorders (e.g. schizophrenia) and neurological diseases (e.g. AD)³⁶. RXFP3 and RXFP4 (formerly known as GPR100 or GPCR142) are homologous class A G protein-coupled receptors with short N-terminal domain. Ligands of RXFP3 or RXFP4 are only limited to endogenous peptides and their analogues, and no natural product or synthetic agonists have been reported to date except for a scaffold of indole-containing derivatives as dual agonists of RXFP3 and RXFP4^{37,50}.

Theses pertinent binding activities of Melafenoxate for relaxin-3/RXFP3 and relaxin-4/RXFP4 receptors are presented in Table 2.

TABLE 2
Binding activity RXFP3 and RXFP4 sites for Melafenoxate

| | % Inhibition at 5*10⁻⁵ M |
|---|---|
| Assay | Melafenoxate |
| RXFP3 (h) (agonist radioligand) | 29.1 |
| RXFP4 (h) (agonist radioligand) | 19.5 |

Melafenoxate is found to weakly bind with RXFP3 and RXFP4 receptors (Study FR095-0024749-Q Eurofins/leadHunter Jun. 25, 2021; unpublished data).

In these assays' compounds were tested in agonist and antagonist mode with the GPCR Biosensor Assays and match to this design:

### Cell Handling

1. CAMP Hunter cell lines were expanded from freezer stocks according to standard procedures.
2. Cells were seeded in a total volume of 20 μL into white walled, 384-well microplates and incubated at 37° C. for the appropriate time prior to testing.
3. cAMP modulation was determined using the DiscoverX HitHunter CAMP XS+assay.

### Gs Agonist Format

1. For agonist determination, cells were incubated with sample to induce response.
2. Media was aspirated from cells and replaced with 15 μL 2:1 HBSS/10 mM Hepes: CAMP XS+ Ab reagent.
3. Intermediate dilution of sample stocks was performed to generate 4× sample in assay buffer.
4. 5 μL of 4× sample was added to cells and incubated at 37° C. or room temperature for 30 or 60 minutes. Vehicle concentration was 1%.

### Gi Agonist Format

1. For agonist determination, cells were incubated with sample in the presence of EC80 forskolin to induce response.
2. Media was aspirated from cells and replaced with 15 μL 2:1 HBSS/10 mM Hepes: cAMP XS+ Ab reagent.
3. Intermediate dilution of sample stocks was performed to generate 4× sample in assay buffer containing 4× EC80 forskolin.
4. 5 μL of 4× sample was added to cells and incubated at 37° C. or room temperature for 30 or 60 minutes. Final assay vehicle concentration was 1%.

### Allosteric Modulation Format

1. For allosteric determination, cells were pre-incubated with sample followed by agonist induction at the EC20 concentration.
2. Media was aspirated from cells and replaced with 10 μL 1:1 HBSS/10 mM Hepes: cAMP XS+ Ab reagent.
3. Intermediate dilution of sample stocks was performed to generate 4× sample in assay buffer.
4. 5 μL of 4× compound was added to the cells and incubated at room temperature or 37° C. for 30 minutes.
5. 5 μL of 4× EC20 agonist was added to the cells and incubated at room temperature or 37° C. for 30 or 60 minutes. For Gi-coupled GPCRs, EC80 forskolin was included.

### Inverse Agonist Format (Gi Only)

1. For inverse agonist determination, cells were pre-incubated with sample in the presence of EC20 forskolin.
2. Media was aspirated from cells and replaced with 15 μL 2:1 HBSS/10 mM Hepes: CAMP XS+ Ab reagent.
3. Intermediate dilution of sample stocks was performed to generate 4× sample in assay buffer containing 4× EC20 forskolin.
4. 5 μL of 4× sample was added to cells and incubated at 37° C. or room temperature for 30 or 60 minutes. Final assay vehicle concentration was 1%.

### Antagonist Format

1. For antagonist determination, cells were pre-incubated with sample followed by agonist challenge at the EC80 concentration.
2. Media was aspirated from cells and replaced with 10 μL 1:1 HBSS/Hepes: CAMP XS+ Ab reagent.
3. 5 μL of 4× compound was added to the cells and incubated at 37° C. or room temperature for 30 minutes.
4. 5 μL of 4× EC80 agonist was added to cells and incubated at 37° C. or room temperature for 30 or 60 minutes. For Gi coupled GPCRs, EC80 forksolin was included.

### Signal Detection

1. After appropriate compound incubation, assay signal was generated through incubation with 20 μL CAMP XS+ ED/CL lysis cocktail for one hour followed by incubation with 20 μL cAMP XS+ EA reagent for three hours at room temperature.
2. Microplates were read following signal generation with a PerkinElmer Envision™ instrument for chemiluminescent signal detection.

### Data Analysis

1. Compound activity was analyzed using CBIS data analysis suite (ChemInnovation, CA).
2. For Gs agonist mode assays, percentage activity is calculated using the following formula:

% Activity=100%×(mean RLU of test sample−mean RLU of vehicle control)/(mean RLU of MAX control−mean RLU of vehicle control).
3. For Gs positive allosteric mode assays, percentage modulation is calculated using the following formula: http://www.eurofinsdiscoveryservices.com Confidential Jun. 25, 2021

5% Modulation=100%×(mean RLU of test sample−mean RLU of EC20 control)/(mean RLU of MAX control−mean RLU of EC20 control).
4. For Gs antagonist or negative allosteric mode assays, percentage inhibition is calculated using the following formula:

% Inhibition=100%×(1−(mean RLU of test sample−mean RLU of vehicle control)/(mean RLU of EC80 control−mean RLU of vehicle control)).
5. For Gi agonist mode assays, percentage activity is calculated using the following formula:

% Activity=100%×(1−(mean RLU of test sample−mean RLU of MAX control)/(mean RLU of vehicle control−mean RLU of MAX control)).
6. For Gi positive allosteric mode assays, percentage modulation is calculated using the following formula:

% Modulation=100%×(1−(mean RLU of test sample−mean RLU of MAX control)/(mean RLU of EC20 control−mean RLU of MAX control)).
7. For Gi inverse agonist mode assays, percentage activity is calculated using the following formula:

% Inverse Agonist Activity=100%×((mean RLU of test sample−mean RLU of EC20 forskolin)/(mean RLU of forskolin positive control−mean RLU of EC20 control)).
8. For Gi antagonist or negative allosteric mode assays, percentage inhibition is calculated using the following formula:

% Inhibition=100%×(mean RLU of test sample−mean RLU of EC80 control)/(mean RLU of forskolin positive control−mean RLU of EC80 control).

For agonist and antagonist assays, data was normalized to the maximal and minimal response observed in the presence of control ligand and vehicle.

For Gi cAMP assays, the following forskolin concentration was used:
RXFP3 CAMP: 20 μM Forskolin
RXFP4 CAMP: 20 μM Forskolin

The following EC80 concentrations were used:
RXFP3 CAMP: 0.0003 μM Relaxin-3
RXFP4 CAMP: 0.01 μM Relaxin-3

### Effect of Melafenoxate (NLS-8) on Memory in a Mouse Model of Alzheimer Disease (AD), the Scopolamine-Induced Amnesia in the Novel Object Recognition (NOR) Test in Mice

### Aim

The scopolamine-induced amnesia in the novel object recognition (NOR) test in mice is a recognized in vivo model of Alzheimer Disease (AD).

The aim of this study is to examine whether Melafenoxate (NLS-8) improves amnesia induced by scopolamine in the NOR test.

For this purpose, two experiments were carried out:
Experiment 1: examination of the effect of NLS-8 at 6 doses on spontaneous locomotor activity.
Experiment 2: examination of the effect of NLS-8 at 3 doses (which do not induce significant alteration in spontaneous locomotor activity) on amnesia induced by scopolamine.

Experiments were carried out on C57BL/6J mice.

### Experiment 1

### Method

The effects of NLS-8 (6 doses) on spontaneous locomotor activity were examined. For this purpose, animals received an injection of NLS-8 (3.125, 6.25, 12.5, 25, 50 and 100 mg/kg) or its vehicle (Control group) (N=6/group) and were placed in an open-field for a 90-min session.

Locomotor activity (distance travelled in the open-field during the first, second and third 30-min period following treatment) was automatically recorded.

### Read-Out: Distance Travelled.

Data analysis: between group comparisons of all groups vs. the Control group: unpaired Student's t test.

### Results

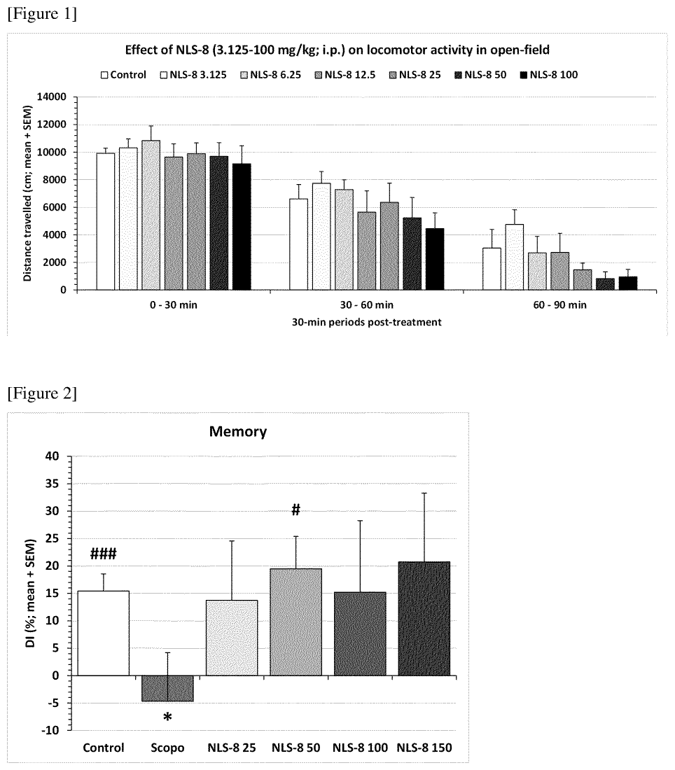

NLS-8 (3.125, 6.25, 12.5, 25, 50 and 100 mg/kg) did not significantly modify the distance travelled (FIG. 1).

### CONCLUSION

NLS-8 (3.125, 6.25, 12.5, 25, 50 and 100 mg/kg) did not significantly modify the locomotor activity and therefore did not induce visible side effect at any dose tested.

Based on this results it was decided to test NLS-8 at 25, 50, 100 and 150 mg/kg in the test of amnesia induced by scopolamine.

### Experiment 2

### Method

In a first step of the experiment, NLS-8 was tested at 25, 50 and 100 mg/kg.

Animals were divided into 6 groups (N=4-10/group).

At T=0 min, they received the following treatments by intraperitoneal (i.p.) route:
Control group: vehicle.
Scopolamine group: scopolamine (1.2 mg/kg).
NLS-8 25 group: NLS-8 (25 mg/kg)+scopolamine (1.2 mg/kg).
NLS-8 50 group: NLS-8 (50 mg/kg)+scopolamine (1.2 mg/kg).
NLS-8 100 group: NLS-8 (100 mg/kg)+scopolamine (1.2 mg/kg).
NLS-8 150 group: NLS-8 (150 mg/kg)+scopolamine (1.2 mg/kg).

At T=30 min following treatment, animals were subjected to a sample trial. They were placed for a 12-min session in a circular experimental box in which two identical objects were presented (50% of animals: two brown cylindrical vials; 50% of animals: two black rectangular columns). At t=120 min following treatments, mice were subjected to a choice trial. They were placed again for a 12-min session in the same experimental box in which two different objects (a vial and a column) were presented: one of the objects (termed as familiar object) presented in the sample trial and a novel object. Exploration of the objects was recorded by an experimenter blind to treatment and unaware of which object was the novel one and which object was the familiar one.

### Read-Outs:

Index of memory:

DI(discrimination index)=100 ×(exploration time of the novel object−exploration time of the familiar object)/(exploration time of the novel object+exploration time of the familiar object).
Index of exploration: N+F=exploration time of the novel object+exploration time of the familiar object.

### Data Analysis:

Within group comparisons: DI vs. 0 (paired Student's t test).
Between group comparisons vs. the Control group and vs. the Scopo group: unpaired Student's t test.

### Results

As shown in FIG. 2, the discrimination index was:
Within group comparisons:
Significantly higher than 0 for the Control group
Not significantly different from 0 for the Scopo group.
Significantly higher than 0 for the NLS-8 50 group.
Non-significantly higher than 0 for NLS-8 25, NLS-8 100 and NLS-8 150 groups.

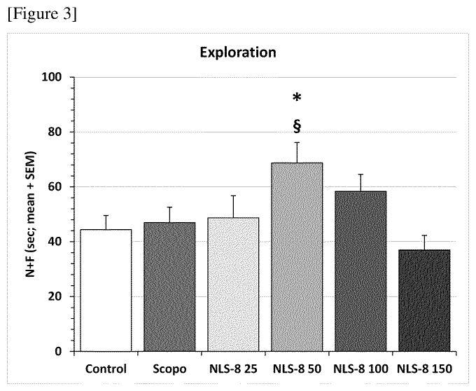

Between group comparisons:
Significantly lower in the Scopo group than in the Control group.
Not significantly different between the Control group and NLS-8 groups.
Not significantly different between the Scopo group and NLS-8 groups.
As shown in FIG. 3, the exploration time of objects in the NLS-8 50 group was higher than that of Control and Scopo groups. All other comparisons vs. the Control group and vs. the Scopo group were not significant.

### CONCLUSIONS

These results suggests that NLS-8 (25-150 mg/kg) can improve amnesia induced by scopolamine and therefore may reduce cognitive symptoms of AD.

### BIBLIOGRAPHY

1. Stephan FK, Zucker I. Circadian rhythms in drinking behavior and locomotor activity of rats are eliminated by hypothalamic lesions. Proc Natl Acad Sci U S A. 1972;69(6):1583-1586. doi:10.1073/pnas.69.6.1583
2. Dijk DJ, Czeisler CA. Paradoxical timing of the circadian rhythm of sleep propensity serves to consolidate sleep and wakefulness in humans. Neurosci Lett. 1994;166(1):63-68. doi:10.1016/0304-3940(94)90841-9
3. Pollak CP, Perlick D. Sleep problems and institutionalization of the elderly. J Geriatr Psychiatry Neurol. 1991;4(4):204-210. doi:10.1177/089198879100400405
4. Satlin A, Volicer L, Stopa EG, Harper D. Circadian locomotor activity and core-body temperature rhythms in Alzheimer's disease. Neurobiol Aging. 1995;16(5):765-771. doi:10.1016/0197-4580(95)00059-n
5. Harper DG, Volicer L, Stopa EG, McKee AC, Nitta M, Satlin A. Disturbance of endogenous circadian rhythm in aging and Alzheimer disease. Am J Geriatr Psychiatry. 2005;13(5):359-368. doi:10.1176/appi.ajgp.13.5.359
6. Goudsmit E, Hofman MA, Fliers E, Swaab DF. The supraoptic and paraventricular nuclei of the human hypothalamus in relation to sex, age and Alzheimer's disease. Neurobiol Aging. 1990;11(5):529-536. doi:10.1016/0197-4580(90)90114-f
7. Sharma A, Sethi G, Tambuwala MM, et al. Circadian Rhythm Disruption and Alzheimer's Disease: The Dynamics of a Vicious Cycle. Curr Neuropharmacol. 2021;19(2):248-264. doi:10.2174/1570159X18666200429013041
8. Saeed Y, Zee PC, Abbott SM. Chapter 24-Clinical neurophysiology of circadian rhythm sleep-wake disorders. In: Levin KH, Chauvel P, eds. Handbook of Clinical Neurology. Vol 161. Elsevier; 2019:369-380. doi:10.1016/B978-0-444-64142-7.00061-8
9. Emet M, Ozcan H, Ozel L, Yayla M, Halici Z, Hacimuftuoglu A. A Review of Melatonin, Its Receptors and Drugs. Eurasian J Med. 2016;48(2):135-141. doi:10.5152/eurasianjmed.2015.0267
10. Muhammad T, Ali T, Ikram M, Khan A, Alam SI, Kim MO. Melatonin Rescue Oxidative Stress-Mediated Neuroinflammation/Neurodegeneration and Memory Impairment in Scopolamine-Induced Amnesia Mice Model. J Neuroimmune Pharmacol. 2019;14(2):278-294. doi:10.1007/s11481-018-9824-3
11. Liu RY, Zhou JN, van Heerikhuize J, Hofman MA, Swaab DF. Decreased Melatonin Levels in Postmortem Cerebrospinal Fluid in Relation to Aging, Alzheimer's Disease, and Apolipoprotein E-ϵ4/4 Genotype1. The Journal of Clinical Endocrinology & Metabolism. 1999;84(1):323-327. doi:10.1210/jcem.84.1.5394
12. Zhou JN, Liu RY, Kamphorst W, Hofman MA, Swaab DF. Early neuropathological Alzheimer's changes in aged individuals are accompanied by decreased cerebrospinal fluid melatonin levels. J Pineal Res. 2003;35(2):125-130. doi:10.1034/j.1600-079x.2003.00065.x
13. Ling ZQ, Tian Q, Wang L, et al. Constant Illumination Induces Alzheimer-Like Damages with Endoplasmic Reticulum Involvement and the Protection of Melatonin. Journal of Alzheimer's Disease. 2009;16(2):287-300. doi:10.3233/JAD-2009-0949
14. Ferguson SA, Rajaratnam SM, Dawson D. Melatonin agonists and insomnia. null. 2010;10(2):305-318. doi:10.1586/ern.10.1
15. Vriend J, Reiter RJ. Breast cancer cells: Modulation by melatonin and the ubiquitin-proteasome system—a review. Mol Cell Endocrinol. 2015;417:1-9. doi:10.1016/j.mce.2015.09.001
16. Slats D, Claassen JAHR, Verbeek MM, Overeem S. Reciprocal interactions between sleep, circadian rhythms and Alzheimer's disease: focus on the role of hypocretin and melatonin. Ageing Res Rev. 2013;12(1):188-200. doi:10.1016/j.arr.2012.04.003
17. American Academy of Sleep Medicine. International Classification of Sleep Disorders. American Acad. of Sleep Medicine; 2014.
18. American Psychiatric Association, American Psychiatric Association, eds. Diagnostic and Statistical Manual of Mental Disorders: DSM-5. 5th ed. American Psychiatric Association; 2013.
19. Organisation mondiale de la Santé. CIM-10: Classification Internationale des Maladies, 10e révision. Published online 1994.
20. Kushida C. Encyclopedia of Sleep. Elsevier Science; 2012. https://books.google.fr/books?id=9UCAMTE7FZgC
21. Lavoie CJ, Zeidler MR, Martin JL. Sleep and aging. Sleep Science and Practice. 2018;2(1):3. doi:10.1186/s41606-018-0021-3
22. Nous A, Engelborghs S, Smolders I. Melatonin levels in the Alzheimer's disease continuum: a systematic review. Alzheimer's Research & Therapy. 2021;13(1):52. doi:10.1186/s13195-021-00788-6
23. Barrón-González M, Rosales-Hernández MC, Abad-Garcia A, et al. Synthesis, In Silico, and Biological Evaluation of a Borinic Tryptophan-Derivative That Induces Melatonin-like Amelioration of Cognitive Deficit in Male Rat. International Journal of Molecular Sciences. 2022;23(6). doi:10.3390/ijms23063229
24. Li Y, Zhang J, Wan J, Liu A, Sun J. Melatonin regulates Aβ production/clearance balance and Aβ neurotoxicity: A potential therapeutic molecule for Alzheimer's disease. Biomedicine & Pharmacotherapy. 2020;132:110887. doi:10.1016/j.biopha.2020.110887
25. Roy J, Tsui KC, Ng J, Fung ML, Lim LW. Regulation of Melatonin and Neurotransmission in Alzheimer's Disease. International Journal of Molecular Sciences. 2021;22(13). doi:10.3390/ijms22136841
26. Hardeland R. Aging, Melatonin, and the Pro-and Anti-Inflammatory Networks. International Journal of Molecular Sciences. 2019;20(5). doi: 10.3390/ijms20051223
27. Shah F, Gupta V. Kleine-Levin syndrome (KLS). In: StatPearls. StatPearls Publishing; 2021.
28. Ambati A, Hillary R, Leu-Semenescu S, et al. Kleine-Levin syndrome is associated with birth difficulties and genetic variants in the TRANK1 gene loci. Proc Natl Acad Sci U S A. 2021;118(12). doi:10.1073/pnas.2005753118
29. Li W, Cai X, Li H-J, et al. Independent replications and integrative analyses confirm TRANK1 as a susceptibility gene for bipolar disorder. Neuropsychopharmacology. 2021;46(6):1103-1112. doi:10.1038/s41386-020-00788-4
30. Hardeland R, Poeggeler B, Srinivasan V, Trakht I, Pandi-Perumal SR, Cardinali DP. Melatonergic drugs in clinical practice. Arzneimittelforschung. 2008;58(1):1-10. doi:10.1055/s-0031-1296459
31. Comai S, Lopez-Canul M, De Gregorio D, et al. Melatonin MT1 receptor as a novel target in neuropsychopharmacology: MT1 ligands, pathophysiological and therapeutic implications, and perspectives. Pharmacological Research. 2019;144:343-356. doi:10.1016/j.phrs.2019.04.015
32. Sulkava S, Muggalla P, Sulkava R, et al. Melatonin receptor type 1A gene linked to Alzheimer's disease in old age. Sleep. 2018;41(7). doi:10.1093/sleep/zsy103
33. Wang X, Sirianni A, Pei Z, et al. The Melatonin MT1 Receptor Axis Modulates Mutant Huntingtin-Mediated Toxicity. J Neurosci. 2011;31(41):14496. doi:10.1523/JNEUROSCI.3059-11.2011
34. Adi N, Mash DC, Ali Y, Singer C, Shehadeh L, Papapetropoulos S. Melatonin MT1 and MT2 receptor expression in Parkinson's disease. Med Sci Monit. 2010;16(2):BR61-67.
35. Farez MF, Mascanfroni ID, Méndez-Huergo SP, et al. Melatonin Contributes to the Seasonality of Multiple Sclerosis Relapses. Cell. 2015;162(6):1338-1352. doi:10.1016/j.cell.2015.08.025
36. Zhang Y, Cook A, Kim J, et al. Melatonin inhibits the caspase-1/cytochrome c/caspase-3 cell death pathway, inhibits MT1 receptor loss and delays disease progression in a mouse model of amyotrophic lateral sclerosis. Neurobiol Dis. 2013;55:26-35. doi:10.1016/j.nbd.2013.03.008
37. Ma S, Smith CM, Blasiak A, Gundlach AL. Distribution, physiology and pharmacology of relaxin-3/RXFP3 systems in brain. Br J Pharmacol. 2017;174(10):1034-1048. doi:10.1111/bph.13659
38. de Ávila C, Chometton S, Ma S, et al. Effects of chronic silencing of relaxin-3 production in nucleus incertus neurons on food intake, body weight, anxiety-like behaviour and limbic brain activity in female rats. Psychopharmacology (Berl). 2020;237(4):1091-1106. doi:10.1007/s00213-019-05439-1
39. Kumar JR, Rajkumar R, Jayakody T, et al. Relaxin' the brain: a case for targeting the nucleus incertus network and relaxin-3/RXFP3 system in neuropsychiatric disorders. Br J Pharmacol. 2017;174(10):1061-1076. doi:10.1111/bph.13564
40. Ganella DE, Ma S, Gundlach AL. Relaxin-3/RXFP3 Signaling and Neuroendocrine Function—A Perspective on Extrinsic Hypothalamic Control. Front Endocrinol (Lausanne). 2013;4:128. doi:10.3389/fendo.2013.00128
41. Chen D, Zhang T, Lee TH. Cellular Mechanisms of Melatonin: Insight from Neurodegenerative Diseases. Biomolecules. 2020;10(8). doi:10.3390/biom10081158
42. Chen BH, Park JH, Lee TK, et al. Melatonin attenuates scopolamine-induced cognitive impairment via protecting against demyelination through BDNF-TrkB signaling in the mouse dentate gyrus. Chem Biol Interact. 2018;285:8-13. doi:10.1016/j.cbi.2018.02.023
43. Chen BH, Ahn JH, Park JH, et al. Effects of Scopolamine and Melatonin Cotreatment on Cognition, Neuronal Damage, and Neurogenesis in the Mouse Dentate Gyrus. Neurochem Res. 2018;43(3):600-608. doi:10.1007/s11064-017-2455-x
44. Konar A, Shah N, Singh R, et al. Protective Role of Ashwagandha Leaf Extract and Its Component Withanone on Scopolamine-Induced Changes in the Brain and Brain-Derived Cells. PLOS ONE. 2011;6(11):e27265. doi:10.1371/journal.pone.0027265
45. Falsafi SK, Deli A, Höger H, Pollak A, Lubec G. Scopolamine administration modulates muscarinic, nicotinic and NMDA receptor systems. PLOS One. 2012;7(2):e32082. doi:10.1371/journal.pone.0032082
46. Budzynska B, Boguszewska-Czubara A, Kruk-Slomka M, et al. Effects of imperatorin on scopolamine-induced cognitive impairment and oxidative stress in mice. Psychopharmacology. 2015;232(5):931-942. doi:10.1007/s00213-014-3728-6
47. Venkatesan R, Subedi L, Yeo EJ, Kim SY. Lactucopicrin ameliorates oxidative stress mediated by scopolamine-induced neurotoxicity through activation of the NRF2 pathway. Neurochemistry International. 2016;99:133-146. doi:10.1016/j.neuint.2016.06.010
48. Birks JS, Harvey RJ. Donepezil for dementia due to Alzheimer's disease. Cochrane Database Syst Rev. 2018;6(6):CD001190. doi:10.1002/14651858.CD001190.pub3
49. Cheng Y, Prusoff WH. Relationship between the inhibition constant (K1) and the concentration of inhibitor which causes 50 per cent inhibition (150) of an enzymatic reaction. Biochem Pharmacol. 1973;22(23):3099-3108. doi:10.1016/0006-2952(73)90196-2
50. Lin L, Lin G, Zhou Q, et al. Design, synthesis and pharmacological evaluation of tricyclic derivatives as selective RXFP4 agonists. Bioorg Chem. 2021;110:104782. doi:10.1016/j.bioorg.2021.104782

1-15. (canceled)

16. A method of prevention and/or treatment of circadian rhythm sleep disorders and/or circadian rhythm sleep neurological diseases and/or any medical condition associated due to sleep deprivation and/or circadian rhythm disruption and/or central disorders of hypersomnolence, comprising the administration of a compound of formula (I) 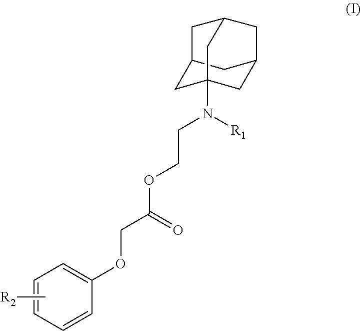
with R₁=H or an acyl group, and
R₂=a halogen atom selected in the group consisting of F, Cl, Br and I, or a pharmaceutically acceptable isomer, salt and/or solvate thereof, to a patient in need thereof.

17. The method of claim 16, wherein R₁=H.

18. The method of claim 16, wherein R₂=Cl.

19. The method of claim 16, wherein the circadian rhythm sleep disorders are selected from the group consisting of Circadian Rhythm Sleep Disorder, Irregular Sleep-Wake Type G47.23 (ICD-10), Circadian Rhythm Sleep Disorder, Irregular Sleep-Wake Type 307.45-3 (DSM-5), Circadian Rhythm Sleep-Wake Disorder, and Irregular Sleep-Wake Rhythm Disorder 307.45-3 (ICSD-3).

20. The method of claim 16, wherein the circadian rhythm sleep neurological diseases are selected from the group of consisting of dementia, Alzheimer's Disease, Huntington's disease, Parkinson's Disease, and Amyotrophic Lateral Sclerosis.

21. The method of claim 16, wherein the circadian rhythm sleep neurological diseases are selected from the group consisting of Angelman syndrome, Williams syndrome, and Smith-Magenis syndrome.

22. The method of claim 16, wherein the central disorders of hypersomnolence are selected from the group of Narcolepsy Type 1, Narcolepsy Type 2, Idiopathic Hypersomnia, Kleine-Levin syndrome, hypersomnia associated with a psychiatric disorder, hypersomnia due to a medical disorder, hypersomnia due to a medication or substance, and insufficient sleep syndrome.

23. The method of claim 16, wherein a therapeutic dose of 0.1 mg/kg/day to 100 mg/kg/day is administrated to the patient in need thereof.

24. The method of claim 16, wherein R₂ is in para position.

25. A method of prevention and/or treatment of circadian rhythm sleep disorders and/or circadian rhythm sleep neurological diseases and/or any medical condition associated due to sleep deprivation and/or circadian rhythm disruption and/or central disorders of hypersomnolence, comprising the administration of a pharmaceutical composition comprising a compound of formula (I) as defined in claim 16 or a pharmaceutically acceptable isomer, salt, and/or solvate thereof, and a pharmaceutically acceptable excipient.

26. The method of claim 25, wherein the circadian rhythm sleep disorders are selected from the group consisting of Circadian Rhythm Sleep Disorder, Irregular Sleep-Wake Type G47.23 (ICD-10), Circadian Rhythm Sleep Disorder, Irregular Sleep-Wake Type 307.45-3 (DSM-5); Circadian Rhythm Sleep-Wake Disorder, and Irregular Sleep-Wake Rhythm Disorder 307.45-3 (ICSD-3).

27. The method of claim 25, wherein the circadian rhythm sleep neurological diseases are selected from the group consisting of dementia, Alzheimer's Disease, Huntington's disease, Parkinson's Disease, and Amyotrophic Lateral Sclerosis.

28. The method of claim 25, wherein the circadian rhythm sleep neurological diseases are selected from the group consisting of Angelman syndrome, Williams syndrome, and Smith-Magenis syndrome.

29. The method of claim 25, wherein the central disorders of hypersomnolence are selected from the group consisting of Narcolepsy Type 1, Narcolepsy Type 2, Idiopathic Hypersomnia, Kleine-Levin syndrome, hypersomnia associated with a psychiatric disorder, hypersomnia due to a medical disorder, hypersomnia due to a medication or substance, and insufficient sleep syndrome.

30. The method of claim 25, wherein the pharmaceutical composition comprises from 1 mg to 800 mg of the compound of formula (I).

31. The method of claim 25, wherein the pharmaceutical composition comprises from 20 mg to 400 mg of the compound of formula (I).

32. The method of claim 25, wherein the pharmaceutical composition is suitable for oral or parenteral administration.

33. The method of claim 25, wherein the pharmaceutical composition is in the form of a solution, a tablet, a capsule, or a transdermal delivery system.

34. The method of claim 33, wherein the pharmaceutical composition is in the form of an injectable solution.
